(19) United States
(12) Patent Application Publication
(10) Pub. No.: US 2012/0046756 A1
(43) Pub. Date: Feb. 23, 2012

(54) TREATMENT OF NASAL CAVITIES WITH STENT HAVING A SOFT OUTER LAYER

(75) Inventors: Yunbing Wang, Sunnyvale, CA (US); Syed F.A. Hossainy, Hayward, CA (US)

(73) Assignee: Abbott Cardiovascular Systems Inc., Santa Clara, CA (US)

(21) Appl. No.: 13/282,404

(22) Filed: Oct. 26, 2011

Related U.S. Application Data
(62) Division of application No. 12/205,736, filed on Sep. 5, 2008.

(51) Int. Cl. A61F 2/04

(52) U.S. Cl. 623/23.7

(57) ABSTRACT

A method of treatment of sinusitis and other related diseases including disposing a radially expandable stent into a nasal passageway and radially expanding the stent in a blocked region of the nasal passageway to expand the blocked region is disclosed. The stent includes a tissue-cushioning portion composed of a hydrogel-forming or elastomeric polymer material to provide cushioning between the stent and nasal tissue and reduce irritation of the nasal tissue.

### CROSS-REFERENCE TO RELATED APPLICATIONS

This application is a divisional of application Ser. No. 12/205,736, filed Sep. 5, 2008, which is incorporated herein by reference.

### BACKGROUND OF THE INVENTION

1. Field of the Invention

This invention relates to methods of treatment of sinusitis and related diseases or conditions by use of a stent in a nasal passageway including an ostium.

2. Background

Sinusitis is an inflammation of mucus membranes in the paranasal sinuses often resulting from infection. The sinuses are hollow cavities around the nose, cheeks and eyes. An illustration is shown in FIG. 1. Specifically, there are four pairs. With reference to FIG. 1, the maxilliary 110 are below the eyes, the frontal 120 are above the eyes, and the ethmoid and sphenoid 130 are located between the eyes. The exact function or purpose of the sinus cavities is unknown. One purpose is believed to be to remove dust from inhaled air. Other purposes may be to lighten the skull, provide vocal resonance, and warming and dehumidification of air. Each of the sinuses produces mucus, and each sinus cavity is connected to the nose which allows for air exchange. The opening to each sinus from the nasal cavity is called an ostium.

The specific structure of the tissue lining the paranasal sinuses is depicted in FIG. 2. The sinuses are lined with a pseudostratified columnar epithelium which also lines the posterior two-thirds of the nasal cavities. FIG. 2 is a representation of the physiology of the surface of the sinus. With reference to the depiction in FIG. 2, there are several layers—a top mucus layer 815 adjacent to the nasal or sinus cavity 850, a bottom mucus layer 818 adjacent to the epithelium 810, a thin basement membrane 820, the lamina propria 830 which is a type of connective tissue found under mucus membrane, and the periosteum 840 which is defined as “a fibrous sheath that covers bones.” Capillaries are found in the basement membrane 820. The epithelium contains four types of cells, one type of which is ciliated cells. The cilia 812 on each ciliated cell beat in unison with cilia on neighboring cells and this action clears particulates from the sinuses. The normal half-life for non-absorbed substances administered to the nasal cavity is about 15 minutes due to the mucociliary clearance moving foreign particles and excess mucus toward the pharynx where the mucus is swallowed. The nasopharynx is behind and above the soft palate, that is the tissue forming the back of the mouth, and which has no bones. The nasopharynx is connected to the nasal cavity. (Sources: G. Porter, F. B. Quinn, Jr., M.D., “Paranasal Sinus Anatomy and Function,” Grand Rounds Presentation, UTMB, Department of Otolaryngology, Jan. 9, 2002, eds F. B. Quinn, Jr., and M. W. Ryan; D. Tomenzoli, Chapter 3: Physiology of the Nose and Paranasal Sinuses,” from Imaging in Treatment Planning for Sinonasal Disease, eds. R. Maroldi and P. Nicolai, Springer-Verlag, Berlin Heidelberg, 2005; P. Van Cauwenberge et al., “Anatomy and Physiology of the Nose and the Paranasal Sinuses,” Immunol Allergy Clin N Am, 24 (2004) 1-17).

Sinus infection may result from a cold, but may also be caused by a fungus, allergies, or medication. Sometimes there are structural abnormalities that lead to problems in the sinus cavities. Inflammation of the mucosal tissue lining the sinuses may block the nasal passageway, and thus may also lead to further bacterial growth due to the inability of the sinus to drain properly.

Many cases of sinusitis are acute, such as when one catches a cold. However, some individuals develop chronic sinusitis. Generally sinusitis is deemed to be chronic if the symptoms exist for six weeks or more. Prevalence of sinusitis is unclear. Some sources state that sinusitis affects about 37 million Americans in the United States (US). However, a 2004 report based on a study performed by the Mayo clinic indicated that the prevalence in one county in Minnesota was 2%.

There are a number of treatments for sinusitis. The medical treatment for sinusitis typically includes a combination of oral antibiotics such as amoxicillin, topical or oral decongestants, steroid nasal sprays, or oral steroids such as prednisone. When medical therapy fails, which is often the case with sinusitis, sinus surgery is an alternative. Surgery alternatives include functional endoscopic sinus surgery (FESS), extended endoscopic frontal sinus surgery, and laser or radiofrequency turbinate reduction. The most common surgery performed today is FESS. The goal of FESS is to improve the drainage of the sinuses by enlarging the nasal passageway (ostia) of the maxillary and frontal sinuses, and opening the ethmoid sinus area by removing the ethmoid air cells under direct visualization. However, FESS itself creates inflammation, which can lead to post-operative fibrosis, stenosis, and/or polyposis that frequently obstructs the newly opened sinuses, requiring the surgeon to re-operate to reopen the nasal passageway.

In 2006, a new surgical treatment for sinusitis called balloon sinuplasty was developed. It involves a small, flexible, sinus balloon catheter that is placed into the nose to reach the sinuses. The sinus balloon catheter is gradually inflated to gently restructure the previously blocked nasal passageway, maintaining the integrity of the sinus lining and restoring normal sinus drainage and function.

### SUMMARY OF THE INVENTION

Various embodiments of the present invention include a method of treatment of sinusitis and other related diseases comprising: disposing a radially expandable stent into a nasal passageway, the stent comprising a plurality of structural elements forming a tubular stent body, wherein the structural elements comprise a tissue-cushioning portion on an abluminal side of the structural elements composed of a hydrogel-forming polymer material and a structural support portion composed of a glassy polymer for providing structural support; and radially expanding the stent in a blocked region of the nasal passageway, wherein the stent expands the blocked region, wherein the tissue-cushioning portion upon swelling of the hydrogel-forming polymer material provides cushioning between the stent and nasal tissue and reduces irritation of the nasal tissue, wherein swelling of the hydrogel-forming polymer material is reduced or prevented for a period of time after disposing the stent in the nasal passageway, wherein a protective coating above the tissue-cushioning portion reduces or prevents the swelling of the hydrogel-forming polymer material for a period of time, wherein the protective coating is formed from a polymer selected the group consisting of a surface-eroding polymer and a bulk-eroding polymer.

### BRIEF DESCRIPTION OF THE DRAWINGS

FIG. 1 illustrates the location of the sinus cavities.

FIG. 2 illustrates the physiology/histology of the nasal and sinus cavity lining.

FIGS. 3A, 3B, and 3C are various depictions of a balloon sinuplasty procedure.

FIG. 4 depicts a stent.

FIGS. 5A-5C depict the deployment of an exemplary stent into one of the ostium of the paranasal sinus cavities.

FIGS. 6A-B depict cross-sections of a stent strut including an abluminal tissue-cushioning layer and a luminal structural support layer.

FIGS. 7A-B depict cross-sections of a stent strut including a tissue-cushioning coating layer around a strut.

FIG. 8 depicts an additional coating layer disposed over the strut of FIGS. 6A-B.

FIG. 9 depicts an additional coating layer disposed over the strut of FIGS. 7A-B.

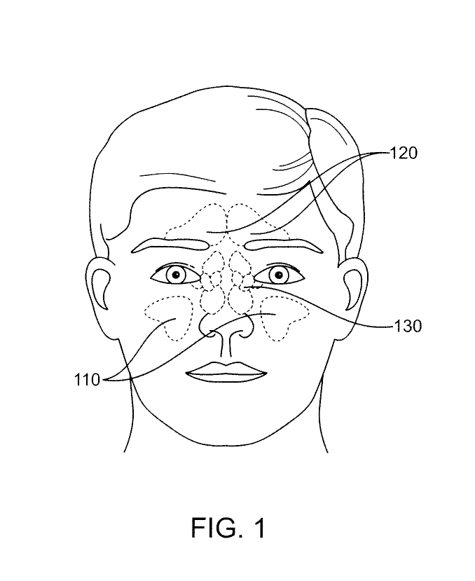

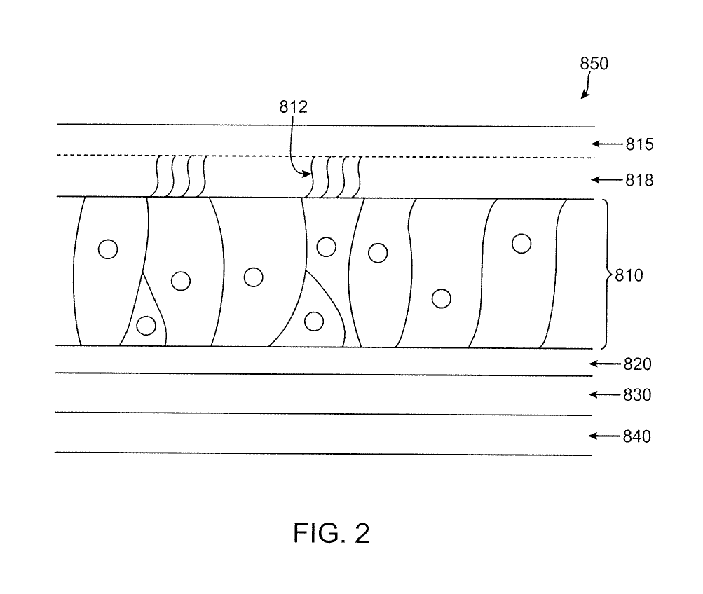

### DETAILED DESCRIPTION OF THE INVENTION

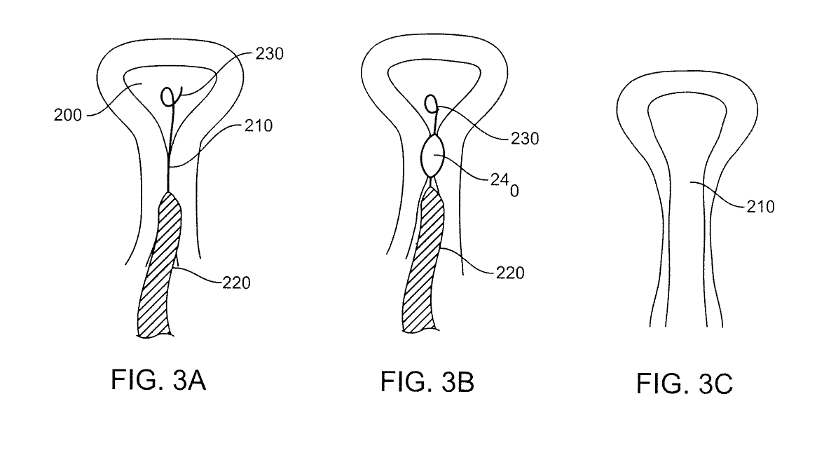

As noted above, in 2006, a new surgical treatment for sinusitis called balloon sinuplasty was developed. Balloon sinuplasty involves the insertion of a sinus balloon into the nose to open up a constricted ostium of the paranasal sinuses. FIG. 3A is a depiction of the ostium 210 to a sinus cavity 200 which is blocked or narrowed due to inflammation. As illustrated in FIG. 3A, a catheter 220 with guidewire 230 is moved through the nose to the blocked or narrowed ostium. When the catheter is in the appropriate location, the balloon 240 disposed on the guidewire 230 attached to catheter 220 is deployed as illustrated in FIG. 3B. Once the blocked or narrowed nasal passageway is opened, the balloon is deflated and removed, leaving the ostium 210 unblocked as illustrated in FIG. 3C.

Various embodiments of the present invention relate to methods of treatment of nasal passageways with a stent. In general, a stent is a type of endoprosthesis that is placed inside the body that is adapted to be implanted in a tubular organ, which in the present invention is a nasal passageway. Such nasal passageways include the ostia of the paranasal sinuses. However, stents may be adapted for treatment for any vessel in the body, including neurological, carotid, vein graft, coronary, aortic, renal, iliac, femoral, popliteal vasculature, and urethral passages. Stents are generally cylindrically shaped devices, which function to hold open and sometimes expand a segment of a tubular organ such as a nasal passageway.

The structure of a stent is typically composed of scaffolding that includes a pattern or network of interconnecting structural elements often referred to in the art as struts or bar arms. The scaffolding can be formed from wires, tubes, or sheets of material rolled into a cylindrical shape. The scaffolding is designed so that the stent can be radially compressed (to allow crimping) and radially expanded (to allow deployment). The present invention is not limited to any particular stent pattern or design. For example, a number of designs are possible, such as, but not limited to, open cell configurations, closed cell configurations, or helical.

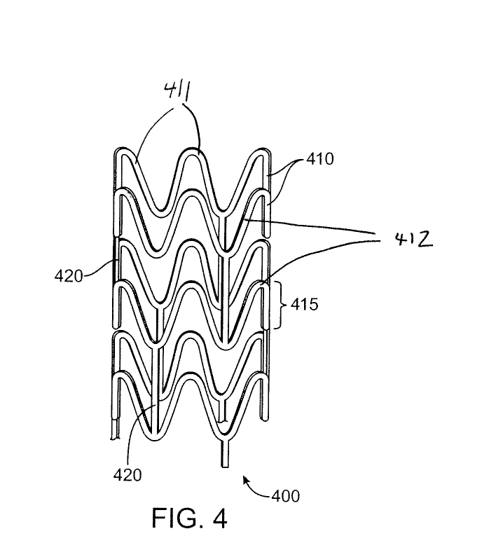

An exemplary embodiment of a stent 400 is depicted in FIG. 4. Stent 400 includes a pattern or network of interconnecting structural elements or struts 410. Stent 400 has struts 410 that form cylindrical rings 415 which are connected by linking struts 420. The embodiments disclosed herein are not limited to any particular stent pattern, and more specifically to the particular stent pattern illustrated in FIG. 4. Stent 400 has a tissue contacting, abluminal, or outer surface 412 and a non-tissue contacting, luminal, or inner surface 411.

A stent pattern, such as, but not limited to the one illustrated in FIG. 4, may be formed on a tube or sheet by laser machining or other methods known in the art. Representative examples of lasers that may be used include, but are not limited to, excimer, carbon dioxide, YAG (yttrium aluminum garnet), or a femtosecond laser. Other methods known in the art, such as but not limited to chemical etching, may be used to cut a stent pattern into the tube.

In some embodiments of the present invention, the stent can be medicated with drugs for the treatment of sinusitis and related diseases or other diseases. In addition, a medicated stent may be provided by a drug coating on a stent surface or incorporating a drug a polymeric coating or layer on a stent. The polymeric body or scaffolding may also serve as a carrier of an active agent or drug.

Various embodiments of the present invention relate to methods of treatment for sinusitis and related diseases utilizing embodiments of stents described below designed to be implanted into a nasal passageway, such as the ostia of the paranasal sinus cavities. The stent can be deployed in a nasal passageway to remove a blockage of the passageway including an ostium, provide a scaffolding function after the blockage has been removed, or both. In particular, embodiments of the present invention include treatment of a nasal passageway with a stent having a soft, elastic, minimally irritating tissue-cushioning portion and a rigid non-tissue contacting portion.

The methods of treatment of the present invention involve the delivery and deployment of the stent in a nasal passageway such as an ostium of the paranasal sinuses. “Delivery” refers to introducing and transporting the stent through the body, in this case introduction through the nose, and maneuvering within the nasal cavity to the region requiring treatment, including a nasal passageway such as an ostium. “Deployment” corresponds to the expanding of the stent once the appropriate passageway is located.

An exemplary, mechanism for delivery and deployment of a stent is to use a catheter, as outlined above in the functional endoscopic sinus surgery (FESS) procedure. In general, the stent is compressed or crimped onto a catheter balloon. Once the end of the catheter is at the appropriate location within a nasal passageway, the stent is then expanded by inflating the balloon. The balloon may then be deflated and the catheter withdrawn, leaving the stent behind to hold open the nasal passageway. In the case of a self-expanding stent, the stent may be secured to the catheter via a constraining member such as a retractable sheath or a sock, which is removed to deploy the stent.

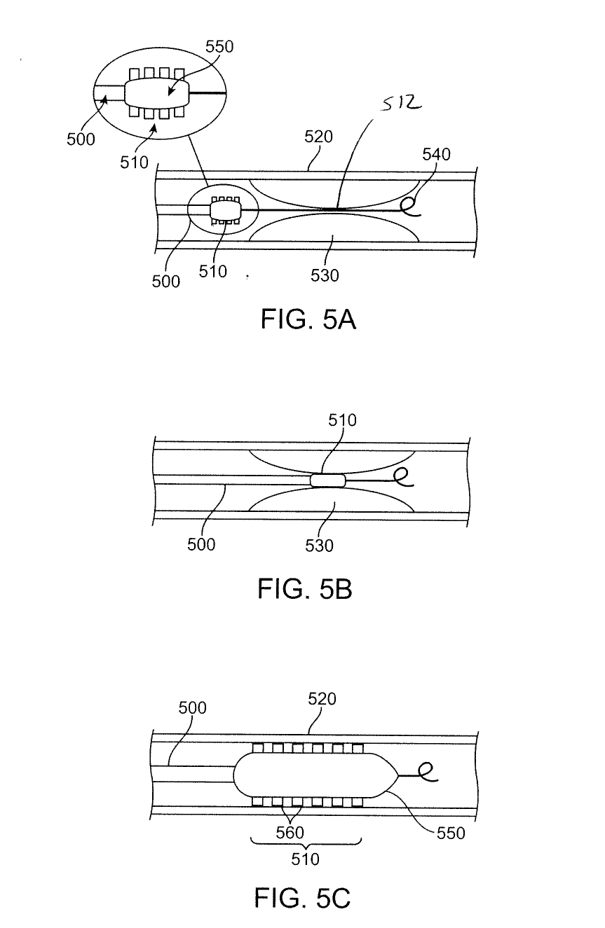

An exemplary depiction of the deployment or implantation of a stent in a nasal passageway such as an ostium is illustrated in FIGS. 5A-5C. FIG. 5A is a pictorial representation of a sinus ostium 520 with a partially blocked nasal passageway 512. A balloon 550 is shown positioned at an end of catheter 500. A stent 510 crimped over balloon 550 is shown approaching the nasal passageway with a tissue blockage 530 in ostium 520 obstructing nasal passageway 512. Guidewire 540 is attached to the end of the catheter 500. FIG. 5B shows stent 510 positioned at tissue blockage 530. FIG. 5C illustrates stent 510 expanded by catheter balloon 550. Struts 560 of stent 510 are pressed against the wall of the ostium (nasal passageway) to remove the blockage nasal passageway 512 of ostium 520. Subsequently, the catheter balloon can be deflated and removed, leaving the stent implanted in the ostium. Although the exemplary embodiment depicted and described in FIGS. 5A-5C is described as an ostium, the passageway is not limited to an ostium.

In the various above mentioned embodiments, the deployment of a stent in a blocked nasal passageway reduces or removes the blockage in the nasal passageway. Thus, in such embodiments, the stent provides a scaffolding function preventing, or helping to prevent, the re-blockage of the nasal passageway. Blockage can be 100% blockage such that no fluid can escape the passageway. A condition may be categorized as a “blockage” even if it is not completely blocked, that is the opening is less than 10%, 10-20%, 20-30%, 30-40%, 40-50%, or greater than 50% of the normal size where normal refers to the typical state where there is no inflammation or other problems. The stent may provide a scaffolding function until removed, if it is not biodegradable, or for a limited time, if it is biodegradable.

In the various embodiments of the present invention, the duration of treatment may be for less than about one (1) month, less than about 3 months, less than about 6 months, or less than about 12 months. At the end of the treatment period, the stent may be removed if it is not bioabsorbable. If bioabsorbable, at the end of the treatment duration, the stent may have degraded to an extent that it can no longer serve a scaffolding function. The stent can completely erode away and/or be swept away from the implant site.

It is believed that the pressure to open the crimped stent and/or expand a blocked nasal passageway is about 2 to about 12 atmospheres. In some embodiments, the pressure of the balloon to open the crimped stent may be 2-4, 4-8, 8-12, or greater than 12 atmospheres.

Exemplary dimensions, without limitation, of the expanded stent include the diameter of the natural ostia of the maxillary sinuses which averages about 2.4 mm, but ranges from 1 mm to 17 mm. Another exemplary dimension, without limitation, is the size of the ostia to the sphenoid sinuses, which ranges from 0.5 to 4 mm in diameter.

In embodiments of the present invention, the method of treatment involves the deployment of a stent that is made from a biodegradable polymers. In many treatment applications, the presence of a stent in a body may be necessary for a limited period of time until its intended function of, for example, functioning as a scaffold to maintain a nasal passageway open, and/or drug delivery is accomplished. Stents fabricated from biodegradable, bioabsorbable, and/or bioerodable polymers can be made so that they completely erode away after the clinical need for them has ended.

The following terms will be used interchangeably: “biologically degradable” (or “biodegradable”), “biologically erodable” (or “bioerodable”), “biologically absorbable” (or “bioabsorbable”), and “biologically resorbable” (or “bioresorbable”), degraded, eroded, absorbed, and dissolved. The above listed terms refer to materials that are capable of being completely, or substantially completely, degraded, dissolved, and/or eroded over time when exposed to physiological conditions, and can be gradually resorbed, absorbed and/or eliminated by the body. Biodegradation is the process of breaking down and eventual absorption and elimination of the material by, e.g., hydrolysis, metabolic processes, oxidation, enzymatic processes, bulk or surface erosion, and the like. Conversely, a “biostable” material refers to a material that is not biodegradable. The time frame after which the stent has completely, or substantially, eroded may be less than about 6 months, less than about 3 months, less than about 2 months, or less than about 1 month. In some embodiments, utilizing a bioabsorbable polymer or other bioabsorbable material, very negligible traces or residue may be left behind.

A stent generally must be capable of withstanding the structural loads, namely radial compressive forces, imposed on the stent as it supports the walls of a nasal passageway. Therefore, a stent must possess sufficient radial strength and rigidity (i.e., sufficiently high modulus) to support lumen walls and maintain such support. Radial strength, which is the ability of a stent to resist radial compressive forces, is due to strength around a circumferential direction of the stent. A sufficiently high modulus also allows the stent to consistently maintain an expanded diameter. Once expanded, the stent must adequately maintain its size and shape throughout its service life despite the various forces that may come to bear on it. For example, a radially directed force may tend to cause a stent to recoil inward.

Crystalline or semi-crystalline polymers that are glassy or have a Tg above body temperature are particularly attractive as stent materials due to their strength and rigidity at physiological conditions. Physiological conditions include, but are limited to, human body temperature, approximately 37° C. For example, polymers such as PLLA are stiff and strong at physiological conditions. Thus, stent formed from a glassy polymer can suitable for supporting the walls of an ostium with a blocked passageway. However, it is expected that a hard, rigid structural element contacting nasal passageway tissue composed of a glassy polymer will cause irritation to the walls of the ostium contacted by the stent. Such irritation can cause adverse effects to a patient including pain, discomfort, and potentially inflammation and bleeding.

Various embodiments of the present invention include implanting a stent in a nasal passageway, the stent having a tissue-cushioning portion that is formed from a elastomeric material or a hydrogel-forming material. In such embodiments, a tissue-cushioning portion can be made from an elastomeric polymer or a polymer capable of forming a hydrogel (hydrogel-forming polymer material) at the physiological conditions in a nasal passageway. The stent additionally has a structural support portion that provides mechanical support of the walls of the nasal passageway. The structural support portion can be composed of a polymer that is glassy at such physiological conditions.

In some embodiments, the tissue-cushioning portion is composed whole or in part of a biodegradable polymer. Additionally, the support portion can be formed in whole or in part of a biodegradable polymer. Both the tissue-cushioning portion and the support portion can be formed from biodegradable polymers so that the stent can be completely absorbed or eroded away from the implant site.

The structural support portion can be formed from a glassy biodegradable polymer including poly(L-lactide) (PLLA), polyglycolide (PGA), poly(L-lactide)-co-polyglycolide (PLGA), poly(ethylene amide) (PEA), and various polyanhydrides. Exemplary polyanhydrides include poly(sebacic acid-hexadecanioic acid anhydride), poly(fumaric-co-sebacic)acid, poly(sebacic acid-1,3-bis(p-carboxyphenoxy)propane anhydride), poly[1,6-bis(carboxyphenoxy)hexane], poly-[trimellitylimidoglycine-co-bis(carboxyphenoxy)hexane], poly[pyromellitylimidoalanine-co-1,6-bis(carboph-enoxy)-hexane], poly[sebacic acid-co-1,6-bis(p-carboxyphenoxy)hexane], and poly[sebacic acid-co-1,3-bis(p-carboxyphenoxy)propane].

As indicated above, the tissue contacting portion can be formed from an elastomeric polymer that exhibits the elastomeric properties at the conditions within a nasal passageway. Such conditions include the human body temperature of about 37° C. An “elastomeric” or “rubbery” polymer refers to a polymer which can resist and recover from deformation produced by force, as in natural rubber. In one embodiment, elastomers or rubbery polymers can be stretched repeatedly to at least twice their original length and, immediately upon release of the stress, return with force to their approximate original length. Elastomeric polymers tend to have a percent elongation at break larger than glassy polymers. The elastomeric polymer can have a Tg below human body temperature or below ambient temperature. Ambient temperature can refer to a temperature between 20° C. and 30° C.

Biodegradable elastomeric polymers for the tissue-cushioning portion include, but are not limited to, polycaprolactone (PCL), poly(trimethylene carbonate) (PTMC), polydioxanone (PDO), poly(4-hydroxy butyrate) (PHB), and poly(butylene succinate) (PBS) and copolymers thereof. Additionally, the elastomeric polymers can include random or alternating copolymers of elastomeric functional units with glassy functional units such as L-lactide and glycolide. In addition, the tissue-cushioning portion can be formed from a block copolymer including elastomeric blocks and glassy blocks. Exemplary block copolymers include PLLA-b-PCL, PLLA-b-PTMC, and PLLA-b-PDO. The degree of elasticity of the polymer can be adjusted by the fraction of the elastomeric block in the polymer.

In additional embodiments, the elastomeric block of a block copolymer can be a copolymer including glycolide (GA) units, added to increase the degradation rate, and an elastomeric group. Such exemplary block copolymers include PLLA-b-P(CL-co-GA), PLGA-b-(PCL-co-GA), and PLLA-b-P(TMC-co-GA).

In additional embodiments, the tissue-cushioning portion can be formed from a hydrogel-forming polymer. Hydrogels are a class of polymer materials that contain a relatively large volume of absorbed water. A hydrogel-forming polymer is made up of a network of polymer chains that is capable of swelling due to absorption of a large volume of water in relation to the weight of the polymer. The crosslinks in the network can be covalent, electrostatic, hydrophobic, or dipole-dipole in character. The ability to swell is facilitated by the hydrophilicity of the polymer. Hydrophilic water-solubilizing groups that can be present include —OH, —COOH, —CONH₂, and CONH.

Since, hydrogels are mostly liquid in composition, they exhibit densities similar to liquids, however, they have the structural coherence of a solid. For example, a hydrogel can contain over 90 wt % water. A hydrogel is a jelly-like material with a high degree of flexibility very similar to natural tissue, due to the significant water content. Thus, a hydrogel tissue-cushioning portion acts as a cushion against the wall of the nasal passageway and reduces or eliminates irritation or discomfort to a patient that can arise from interaction of the stent with tissue of the nasal passageway.

In the embodiments of the present invention, the hydrogel-forming polymer material for the tissue-cushioning portion can be biostable or biodegradable. In some embodiments, the hydrogel-forming material for the tissue-cushioning portion can be formed from a crosslinked hydrophilic polymer such as polyethylene glycol (PEG), which is a water soluble, biostable polymer. Although PEG is a water soluble polymer, the PEG hydrogel-forming polymer material is insoluble. The insolubility is due to physical or chemical crosslinkage of the hydrophilic polymer chains.

In another embodiment, the tissue-cushioning portion can be formed from a biodegradable hydrogel-forming material. Embodiments of such a materials are hyaluronic acid (HA) hydrogel or a HA-poly(ethylene oxide) (PEO) hydrogel. Biomed. Mater. 1 116-123 (2006).

In further embodiments, the tissue-cushioning portion can be formed from a biodegradable hydrogel including a block copolymer having a hydrophilic component and a degradable hydrophobic component. In such embodiments, the hydrophilic component can be, but is not limited to, HA and PEG. The hydrophobic component can include an elastomeric or glassy polymer including PCL, PDO, PHB, PTMC, PBS, PLLA, PGA, PLGA, and poly(DL-lactide) (PDLLA). Exemplary block copolymers include PEG-b-PCL, PEG-b-PLLA, HA-b-PCL, and HA-b-PLLA. The degree of swelling, and thus the degree of cushioning, can be modified by adjusting the fraction of the hydrophilic component.

In additional embodiments, the hydrogel-forming material can be a random or alternating copolymer having a hydrophilic component and a hydrophobic component. For example, the hydrophilic component can be 2-hydroxyethyl methacrylate (HEMA) and the hydrophobic component can be caprolactone, the exemplary copolymer being poly(2-hydroxyethyl methacrylate)-co-polycaprolactone (P(HEMA-co-CL)). Materials Science Forum Vols. 455-456 (2004) pp. 425-428.

In other embodiments, the tissue-cushioning portion can be formed from a hydrogel-forming material including a graft polymer having a hydrophilic component with a grafted hydrophobic component. An exemplary graft polymer includes poly(2-hydroxyethyl methacrylate)-graft-poly(L-lactide). Ibid.

In certain embodiments, the structural elements of the stent can include an abluminal layer and a luminal layer. In these embodiments, the abluminal layer can be formed from an elastomeric or hydrogel-forming polymer and the luminal layer can be formed from a glassy polymer. The abluminal layer corresponds to the tissue-cushioning portion the structural support portion corresponds to the luminal layer.

FIGS. 6A-B depict cross-sections of a stent strut 600 normal to and parallel to an axis of strut 600, respectively. Strut 600 has an abluminal or tissue-cushioning layer 602 with a tissue-cushioning surface 608 and a luminal or structural support layer 604 with a luminal surface 610. Tissue-contacting surface contacts a wall of a nasal passageway when a stent is deployed. Line 606 depicts the boundary between the layers.

Abluminal layer 602 can be a polymer hydrogel-forming material or elastomeric polymer and luminal layer 604 is formed from a glassy polymer. A thickness Tc of the abluminal layer and a thickness Ts of the luminal layer can be the same or different. Ts generally is large enough to provide mechanical support of the nasal passageway walls. Tc may be adjusted to provide sufficient cushioning between the tissue-cushioning surface and the walls of the nasal passageway.

In another embodiment of a stent for deployment in a nasal passageway, the stent can have a coating layer disposed above the abluminal, luminal, and side wall surfaces of a stent body or scaffolding. The coating layer can be an elastomeric or hydrogel-forming polymer. The tissue-cushioning portion corresponds to an abluminal side of the coating layer and the stent body is the structural support portion of the stent. “Above” refers to over, but not necessarily in contact with. In some embodiments, the coating is disposed over an entire surface of the body or scaffolding, i.e., a luminal surface, abluminal surface, and sidewall surfaces of the structural elements of the stent.

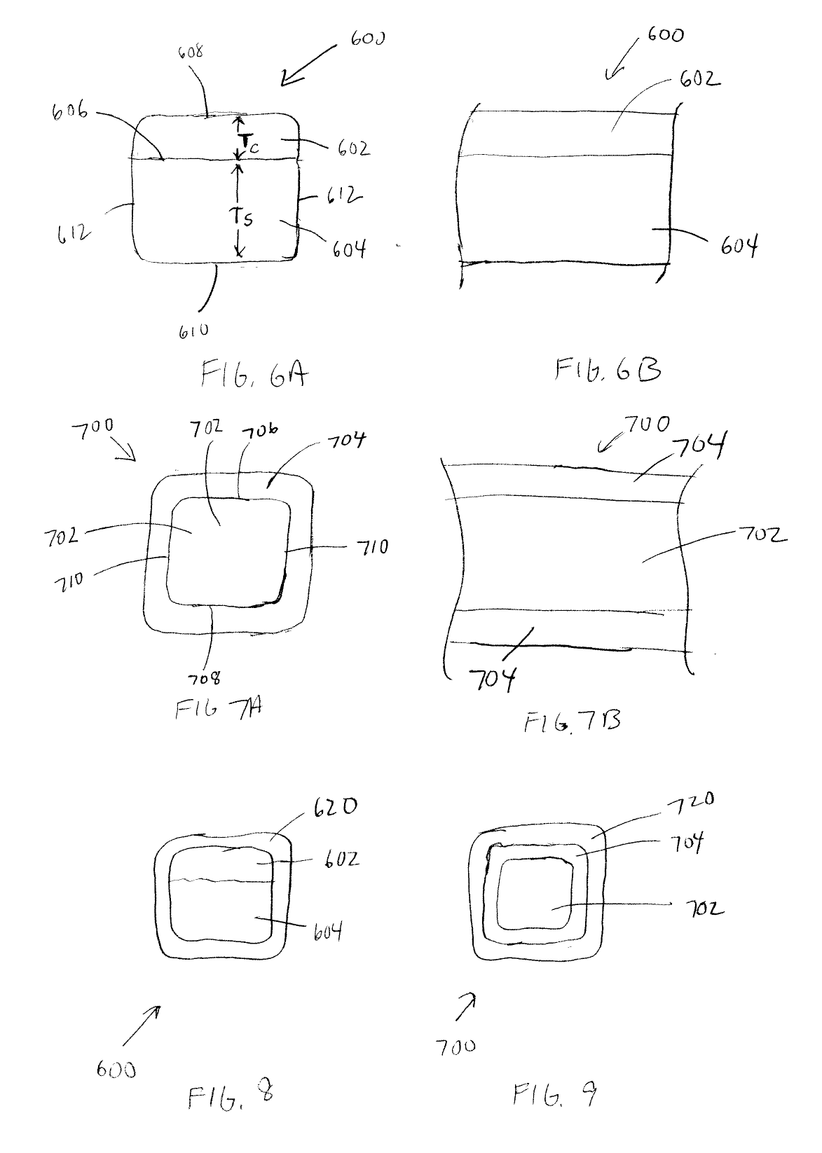

FIGS. 7A-B depict cross-sections of a stent strut 700 normal to and parallel to an axis of a strut 700, respectively. Strut 700 includes a body or structural support portion 702 with a hydrogel-forming or elastomeric coating layer 704 disposed over an abluminal surface 706, a luminal surface 708, and sidewall surfaces 710 of body 702.

In further embodiments, the stent can also include a drug or active agent to treat sinusitis and other paranasal sinus conditions. In some embodiments, the drug can be incorporated in the tissue-cushioning portion such in an abluminal layer or coating layer. For example, the drug can be dispersed in abluminal layer 602 shown in FIGS. 6A-B or in coating layer 704 shown in FIG. 7A-B. In other embodiments, the drug can be dispersed in the glassy polymer of the structural support portion or body of the stent, such as luminal layer 604 in FIGS. 6A-B or body 702 in FIGS. 7A-B.

In additional embodiments, the stent can further include an additional coating layer that includes a drug. For example, FIG. 8 depicts an additional coating layer 620 disposed over strut 600 of FIGS. 6A-B. Similarly, FIG. 9 depicts an additional coating layer 720 disposed over strut 700 of FIGS. 7A-B. The additional medicated coating layer can be a hydrogel or an elastomer with a drug dispersed therein. The mechanism of drug release can be due to diffusion through the polymer or to degradation. The duration of drug delivery may be less than about 1 day, about 1 day to about 1 week, about 1 week to about 1 month, about 1 month to about 3 months, about 3 months to about 6 months, about 6 months to about 12 months, or more than 12 months.

In some embodiments, a stent having a tissue-cushioning portion formed from a hydrogel-forming polymer can be fabricated and inserted into a nasal passageway in a swelled state. In other embodiments, a stent having a tissue-cushioning portion formed from a hydrogel-forming polymer can be fabricated and inserted into a nasal passageway in an unswelled or partially swelled state. In one embodiment, the an unswelled hydrogel-forming polymer swells as the stent is being transported through the nasal passageway to a target implantation region. A partially swelled tissue-cushioning portion absorbs additional moisture in the nasal passageway. In this embodiment, moisture in the nasal passageways is absorbed by the hydrogel-forming polymer causing in it to swell and form a hydrogel.

In alternate embodiments, the moisture absorption of the hydrogel-forming portion can be limited or delayed as the stent is delivered and after the stent is deployed. In an embodiment, the absorption can be limited or prevented for a selected period of time or until a particular point in the delivery and deployment process. In such embodiments, moisture absorption can be limited or prevented by a protective layer disposed over at least a part of the surface of the tissue-cushioning portion of the stent. For example, referring to FIG. 8, coating layer 620 can be a protective layer that protects hydrogel-forming tissue contacting portion 602 from exposure to moisture. For example, referring to FIG. 9, coating layer 720 can be a protective layer that protects a hydrogel-forming tissue contacting portion 704 from exposure to moisture.

In these embodiments, the protective layer can be composed of a water soluble polymer such as PEG or poly(vinyl acetate). The amount of time the protective layer can limit or prevent swelling can be adjusted, for example, by the thickness of the protective coating.

In other embodiments, the protective layer can be a biodegradable polymer. In such embodiments, the biodegradable polymer can be a bulk eroding polymer or a surface eroding polymer. In general, bulk eroding polymers allow penetration of moisture within a polymer sample, so that degradation or erosion can occur throughout the sample. Surface eroding allow little nor not water penetration into a sample so that mass loss occurs completely or primarily at the surface of the sample.

With a bulk eroding protective layer, moisture can diffuse through the protective layer to the hydrogel-forming material. Thus, the swelling of the hydrogel-forming material can occur gradually or over a longer time frame during delivery, deployment, and after deployment. Alternatively, with a surface eroding polymer protective layer, the swelling of the tissue-cushioning portion is expected to occur over a much shorter time frame. The swelling coincides approximately with complete erosion of the surface layer that exposes the surface or portion thereof of the tissue-cushioning portion. Exemplary bulk eroding polymers include the biodegradable polyesters disclosed above. Exemplary surface eroding polymers include various types of polyanhydrides.

In further embodiments, the swelling of a hydrogel-forming tissue contacting portion can be limited or prevented during delivery, deployment, and after deployment by a sheath disposed over the stent. The sheath can be composed of a biostable or biodegradable polymer. The sheath can be removed during delivery, deployment, or after deployment to allow swelling or additional swelling of the tissue contacting portion.

The abluminal tissue-cushioning layer and luminal support layer can be formed through co-extrusion or by a coating method. Coextrusion refers to the process of extruding two or more materials through a single die with two or more orifices arranged so that the extrudates from the separate orifices merge and adhere together into a laminar structure before cooling or chilling. Each material can be fed to the die from a separate extruder into separate orifices. In the case of tubing coextrusion, the die can include concentric circular slits through which the different materials exit to form a tube with two or more layers. With respect to the present invention, a two-layer tube can be formed by co-extrusion, an outer layer composed of a hydrogel-forming polymer or elastomer and an inner layer composed of a glassy polymer. A stent pattern can then be formed in the tube by laser cutting.

Alternatively, an elastomer or hydrogel-forming polymer layer can be formed over an abluminal surface of a stent made of a glassy polymer using a coating technique such as spray coating, dip coating, cast coating, or non-solvent coating using supercritical carbon dioxide as a medium. Spray, dip, and cast coating involve generally forming a coating material that includes a solution or dispersion of the coating polymer in a solvent. The coating material is then applied to the stent surface followed by removal of the solvent through evaporation, resulting in formation of a polymer coating. In order to reduce or prevent swelling, an organic solvent is used that is free or substantially free of moisture.

After the implantation, the stent supports the walls of and maintains the opening of the nasal passageway. The degradable portions of the stent are expected to be gradually absorbed after implantation and are drained or swept away after the inflamed/infected nasal passageway is fully healed and regains its original function. A hydrogel that is biostable is insoluble, however, can also be drained or swept away. The degradation time of the stent can be designed to be from 1-2 months, 3-6 months, 6-12 months, or 12 months to 2 years.

The phrase “sinusitis and other related diseases” will encompass, but is not limited to, the following diseases, or conditions: postoperative paranasal sinus inflammation due to functional endoscopic sinus surgery (FESS), acute sinusitis, chronic sinusitis, allergic rhinitis, rhinosinusitis, sinusitis that recurs after FESS, upper respiratory tract infections, otitis media, bronchitis, bronchiolitis, asthma, tonsillitis, and other chronic diseases of the tonsils and adenoids, adenitis, nose surgery, laryngitis, tracheitis, nasal and sinus polyposis, neoplasms of the large and small airways, and nasal, sinus, or nasopharynx tumors such as nasopharyngeal carcinoma, plasmacytomas, inverted papillomas, rhabdomyosarcomas, squamous cell carcinomas, and lymphomas, when they involve the sinuses, or nasal passage.

The term “sinusitis and other related diseases” is intended to encompass diseases within the paranasal sinuses and/or nasal cavity, and/or nasopharynx, as well as diseases that originate in the paranasal sinuses, and/or nasal cavity, and/or nasopharynx. The term “treatment of sinusitis and other related diseases” will refer to treatment of at least one disease or condition from those defined as “sinusitis and other related diseases.”

The therapeutic agents used in the thin drug coating layer include various anti-inflammatory agents, anti-infective agents, and combinations thereof. Anti-inflammatory agents generally include steroidal and nonsteroidal anti-inflammatory agents. Anti-infective agents generally include antibacterial agents, antifungal agents, antiparasitic agents, antiviral agents, and antiseptics. In some embodiments, the stent utilized in the method of treatment may include drugs from multiple therapeutic classes, and/or multiple drugs from one or more therapeutic classes, or any combinations thereof. Some of the specific drugs are outlined below.

Examples of steroidal anti-inflammatory agents that may be used in the coating layer include 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, any of their derivatives, and combinations thereof.

Examples of nonsteroidal anti-inflammatory agents include COX-1 and COX nonspecific inhibitors (e.g., salicylic acid derivatives, aspirin, sodium salicylate, choline magnesium trisalicylate, salsalate, diflunisal, sulfasalazine and olsalazine; para-aminophenol derivatives such as acetaminophen; indole and indene acetic acids such as indomethacin and sulindac; heteroaryl acetic acids such as tolmetin, dicofenac and ketorolac; arylpropionic acids such as ibuprofen, naproxen, flurbiprofen, ketoprofen, fenoprofen and oxaprozin), and selective COX-2 inhibitors (e.g., diaryl-substituted furanones such as rofecoxib; diaryl-substituted pyrazoles such as celecoxib; indole acetic acids such as etodolac and sulfonanilides such as nimesulide).

Examples of antibacterial agents that may be suitable for use in the coating layer include, but are not limited to, aminoglycosides, amphenicols, ansamycins, .beta.-lactams, lincosamides, macrolides, nitrofurans, quinolones, sulfonamides, sulfones, tetracyclines, vancomycin, and any of their derivatives, or combinations thereof.

Examples of antifungal agents suitable for use in the coating layer include, but are not limited to, allylamines, imidazoles, polyenes, thiocarbamates, triazoles, and any of their derivatives. In one variation, imidazoles are the preferred antifungal agents. Antiparasitic agents that may be employed include such agents as atovaquone, clindamycin, dapsone, iodoquinol, metronidazole, pentamidine, primaquine, pyrimethamine, sulfadiazine, trimethoprim/sulfamethoxazole, trimetrexate, and combinations thereof.

Examples of antiviral agents suitable for use in the coating layer include, but are not limited to, acyclovir, famciclovir, valacyclovir, edoxudine, ganciclovir, foscamet, cidovir (vistide), vitrasert, formivirsen, HPMPA (9-(3-hydroxy-2-phosphonomethoxypropyl)adenine), PMEA (9-(2-phosphonomethoxyethyl)adenine), HPMPG (9-(3-Hydroxy-2-(Phosphonomet-hoxy)propyl)guanine), PMEG (9-[2-(phosphonomethoxy)ethyl]guanine), HPMPC (1-(2-phosphonomethoxy-3-hydroxypropyl)-cytosine), ribavirin, EICAR (5-ethynyl-1-beta-D-ribofuranosylimidazole-4-carboxamine), pyrazofurin (3-[beta-D-ribofuranosyl]-4-hydroxypyrazole-5-carboxamine), 3-Deazaguanine, GR-92938X (1-beta-D-ribofuranosylpyrazole-3,4-dicarboxamide), LY253963 (1,3,4-thiadiazol-2-yl-cyanamide), RD3-0028 (1,4-dihydro-2,3-Benzodithiin), CL387626 (4,4′-bis[4,6-d].beta.-aminophenyl-N-,N-bis(2-carbamoylethyl)-sulfonilimino]-1,3,5-triazin-2-ylamino-biphenyl-2-,2′-disulfonic acid disodium salt), BABIM (Bis[5-Amidino-2-benzimidazoly-1]-methane), NIH351, and combinations thereof.

The “glass transition temperature,” T_{g}, is the temperature at which, when observed on the same time frame, the amorphous domains of a polymer change from a brittle, vitreous state to a solid deformable state (or rubbery state). In other words, the T_{g} corresponds to the temperature where the onset of segmental motion in the chains of the polymer occurs. The measured T_{g} of a given polymer can be dependent on the heating rate and can be influenced by the thermal history, and potentially pressure history, of the polymer, as well as potentially the pressure at which the measurement is made.

“Treatment” will refer to (i) preventing the disease or condition from occurring in an individual who may be predisposed to the disease or condition, but has not yet been diagnosed as having it; (ii) inhibiting the disease or condition, i.e. arresting its development; or (iii) relieving the disease or condition, i.e. causing regression of the disease or condition, or (iv) treating the symptoms of the disease or condition.

“Drug,” “active agent,” or “therapeutic agent,” which will be used interchangeably, refers to any substance that, when administered in a therapeutically effective amount to an individual suffering from a disease or condition, has a therapeutic beneficial effect on the health and well-being of the individual (animal, including a human). A therapeutic beneficial effect on the health and well-being of an individual includes, but it not limited to: (1) curing the disease or condition; (2) slowing the progress of the disease or condition; (3) causing the disease or condition to retrogress; or, (4) alleviating one or more symptoms of the disease or condition.

As used herein, a drug also includes any substance that when administered to an individual, known or suspected of being particularly susceptible to a disease or condition, in a prophylactically effective amount, has a prophylactic beneficial effect on the health and well-being of the patient. A prophylactic beneficial effect on the health and well-being of an individual includes, but is not limited to: (1) preventing or delaying on-set of the disease or condition in the first place; (2) maintaining a disease or condition at a retrogressed level once such level has been achieved by a therapeutically effective amount of a substance, which may be the same as or different from the substance used in a prophylactically effective amount; or, (3) preventing or delaying recurrence of the disease or condition after a course of treatment with a therapeutically effective amount of a substance, which may be the same as or different from the substance used in a prophylactically effective amount, has concluded.

In some cases, a compound may be classified as a drug based upon appropriate in-vitro experiments. In some cases, a compound may be classified as drug based upon clinical or experimental data in which the individuals evaluated were not necessarily “patients,” or in other words, individuals who were not either suffering from, or known or suspected of being particularly susceptible to, a disease or condition for which the compound is administered to treat. One example of such class of individuals is healthy, normal individuals.

The term “drug” also encompasses vitamins and minerals such as those contained in a typical vitamin tablet. The term “drug” also encompasses pharmaceutically acceptable, pharmacologically active derivatives of those drugs specifically mentioned herein, including, but not limited to, salts, esters, amides, prodrugs, active metabolites, analogs, and the like.

“Ostium” or “ostia,” as used herein, refer to the ostia connecting the nasal cavity and the sinus cavities, as opposed to other ostia in the body, unless the context clearly dictates otherwise.

“Nasal passageway,” as used herein, refers to a passageway or opening that can benefit from stenting, in the nose, nostril, nasopharynx, nasal cavity, meatus, or paranasal sinuses, including the ostia connecting the nasal cavity and the paranasal sinuses.

“Degradation time” refers to the time for a stent implanted in a vessel to completely absorb in vivo. “Degradation time” can also refer to the time for a stent to completely absorb in vitro.

“Physiological conditions” refer to conditions to which an implant is exposed within the body of an animal (e.g., a human). Physiological conditions include, but are not limited to, “normal” body temperature for that species of animal (approximately 37° C. for a human) and when appropriate, an aqueous environment of physiologic ionic strength, pH and enzymes. In some cases, the body temperature of a particular animal may be above or below what would be considered “normal” body temperature for that species of animal. For example, the body temperature of a human may be above or below approximately 37° C. in certain cases. The scope of the present invention encompasses such cases where the physiological conditions (e.g., body temperature) of an animal are not considered “normal.”

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true spirit and scope of this invention.

What is claimed is:

1. A method of treatment of sinusitis and other related diseases comprising:
disposing a radially expandable stent into a nasal passageway, the stent comprising a plurality of structural elements forming a tubular stent body, wherein the structural elements comprise a tissue-cushioning portion on an abluminal side of the structural elements composed of a hydrogel-forming polymer material and a structural support portion composed of a glassy polymer for providing structural support; and
radially expanding the stent in a blocked region of the nasal passageway, wherein the stent expands the blocked region, wherein the tissue-cushioning portion upon swelling of the hydrogel-forming polymer material provides cushioning between the stent and nasal tissue and reduces irritation of the nasal tissue,
wherein swelling of the hydrogel-forming polymer material is reduced or prevented for a period of time after disposing the stent in the nasal passageway,
wherein a protective coating above the tissue-cushioning portion reduces or prevents the swelling of the hydrogel-forming polymer material for a period of time, wherein the protective coating is formed from a polymer selected the group consisting of a surface-eroding polymer and a bulk-eroding polymer.

2. The method of claim 1, wherein the protective coating is formed from a surface-eroding polymer, wherein the protective coating prevents swelling until erosion of the protective layer exposes a portion of the tissue-cushioning portion.

3. The method of claim 2, wherein the surface eroding polymer comprises a polyanhydride.

4. The method of claim 1, wherein the protective coating is formed from a bulk-eroding polymer.

5. The method of claim 4, wherein the bulk eroding polymer comprises a biodegradable polyester.

6. The method of claim 1, wherein the tissue-cushioning portion comprises an abluminal layer and the structural support portion comprises a luminal layer of the structural elements.

7. The method of claim 1, wherein the hydrogel-forming polymer material is unswelled when the stent is disposed in the nasal passageway.

8. The method of claim 1, wherein the tissue-cushioning portion and the structural support portion are biodegradable, the stent holding open the region for a period of time followed by complete erosion of the stent.

9. The method of claim 1, wherein the hydrogel-forming polymer is selected from the group consisting of PEG, HA, HA-PEG, and P(HEMA-co-PCL).

10. The method of claim 1, wherein the hydrogel-forming polymer is a block copolymer of PEG and a polymer selected from the group consisting of PCL, PDO, PTMC, PHB, PBS, PLLA, PGA, PLGA, PDLLA, P(GA-co-CL), P(GA-co-DO), P(GA-co-TMC), and P(GA-co-BS).

11. The method of claim 1, wherein the hydrogel-forming polymer comprises a therapeutic agent.
